(19) United States
(12) Patent Application Publication
(10) Pub. No.: US 2025/0304927 A1
(43) Pub. Date: Oct. 2, 2025

(54) PRO-PEPTIDE VARIANTS FOR MODULATING ACTIVITY OF TRANSGLUTAMINASES

(71) Applicant: CURIE CO. INC., Durham, NC (US)

(72) Inventors: Erika M. MILCZEK, Durham, NC (US); Will SHINDEL, Durham, NC (US); Christian KASEY, Cary, NC (US)

(21) Appl. No.: 18/866,869

(22) PCT Filed: May 19, 2023
(86) PCT No.: PCT/US2023/067261

§ 371 Date: Nov. 18, 2024

Related U.S. Application Data
(60) Provisional application No. 63/344,392, filed on May 20, 2022.

(51) Int. Cl. C12N 9/10

(52) U.S. Cl. CPC C12N 9/1044; C12Y 203/02013

(57) ABSTRACT

Pro-peptide variants useful in modulating the activity of transglutaminases are disclosed herein.

### CROSS-REFERENCE TO RELATED APPLICATIONS

This application claims the benefit of priority to U.S. Provisional Application No. 63/344,392, filed May 20, 2022, which is hereby incorporated by reference in its entirety.

### STATEMENT REGARDING FEDERALLY SPONSORED RESEARCH

This invention was partially made with government support under Grant No. 2026057, awarded by the National Science Foundation. The government has certain rights in this invention.

### INCORPORATION BY REFERENCE

The sequence listing provided in the file named SequenceListing.xml with a size of 6,605 bytes, which was created on May 18, 2023, and which is filed herewith, is incorporated by reference in its entirety.

### FIELD

The field pertains to pro-peptide variants useful in modulating functional activity of transglutaminases such as, microbial transglutaminases.

### BACKGROUND

Transglutaminases (EC2.3.2.13) are a family of enzymes that catalyze crosslinking between the γ-carboxyamide group in glutamine residues (acyl donors) and a variety of primary amines (acyl acceptors), including the amino group of lysine. Transglutaminases can be found throughout all groups of organisms including plants, animals, and microbes. Transglutaminases in animals, for example, include blood coagulation factor XIII, which is a multi-domain protein and depends on calcium for regulation of enzyme function. Microbial transglutaminases, on the other hand, have only one single domain and do not depend on calcium for activity, i.e., transglutaminases of microbial origin are calcium-independent. Thus, microbial transglutaminases represent a major advantage for their practical use.

Commercially available transglutaminase is produced by fermentation of Streptomyces mobaraensis. transglutaminase is expressed as an inactive zymogen having a pro-peptide sequence at the N-terminus of the mature domain. The active enzyme is produced by removing the pro-peptide, i.e., the pro-domain, prosequence or proregion, by proteolytic processing to afford the mature domain. Thus, the pro-peptide can be regarded as serving a regulatory function while the mature domain serves a catalytic function.

Pro-peptides generally are recognized to have four major functions: 1) pro-peptides can function as intramolecular chaperones or folding assistants by determining the three-dimensional structure of a protein; 2) pro-peptides can function as inhibitors or activation peptides; 3) pro-peptides can direct protein sorting into specific cellular compartments or extra-cellular space and 4) pro-peptides can mediate the precursor interaction with other molecules (such as peptides, proteins, and polysaccharides) or supramolecular structures (e.g., cell walls). A single pro-peptide can perform several or even all these functions.

Due to its poor stability in solution, mature transglutaminase is typically formulated as a powder for commercial application and taken into solution or slurry at the time of use for crosslinking food protein. It was recently shown that in solution the enzyme may act on itself, resulting in the formation of transglutaminase-crosslinked aggregates, greatly reducing its enzymatic activity with exogenous proteins and peptides (Böhme et al., Amino Acids, 2019 52 (2): 313-326)

It has been demonstrated that pro-peptides can modulate protein functional activity irrespective of the specific role or mode of action. They make it possible to substantially alter biological properties of proteins without cardinal changes in major functional (e.g., catalytic) domains of molecules. This appears to be a property of pro-peptides that allows them to regulate protein activity at the post-translational level and to function as specific evolutionary modules providing for functional variation of protein molecules.

The pro-peptide is needed for both proper folding of transglutaminase and intracellular inhibition to prevent toxicity of mature transglutaminase. It has been shown that binding of the pro-peptide of transglutaminase can be altered, e.g., destabilized, through site-directed mutagenesis. Specifically, research has shown that the binding affinity of the pro-peptide to the mature transglutaminase can be modified through site-directed mutagenesis to have weaker inhibitory properties (e.g., Rickert et al., Protein Science, 2016 Feb. 25 (2): 442-455).

WO 2016/170447, published Oct. 27, 2016, discloses recombinant transglutaminases wherein the engineered transglutaminase polypeptides comprise one or more modifications in the pro-domain regions to modulate the interaction between the pro-domain and the enzyme domain of the transglutaminases. It should be noted that the pro-domain sequence of wild-type transglutaminase from Streptomyces mobaraensis corresponding to SEQ ID NO: 1 in that publication, and also shown elsewhere in that publication, such as in FIG. 3B, appear to be incorrect in that the disclosed sequence is missing an aspartic acid at the N-terminus. Thus, since the aspartic acid is missing, the sequence starts with asparagine.

Efforts to mutate pro-peptide residues have focused on residues that were predicted to contact the active site cleft. However, it has been shown that such predictions do not necessarily result in beneficial mutations.

Accordingly, it is believed that many of residues involved in pro-peptide binding, as well as beneficial mutations at any given site, have yet to be identified. Disclosed herein are new pro-peptide variants capable of modulating transglutaminase activity.

### SUMMARY

Disclosed herein are pro-peptide variants of a transglutaminase pro-peptide. The pro-peptide variants disclosed herein increase pH responsiveness of mature transglutaminase, have increased binding affinity to mature transglutaminase, have decrease binding affinity to mature transglutaminase, increase inhibition of mature transglutaminase, increased stability of mature transglutaminase, and/or increase yield of mature transglutaminase.

In a first embodiment, there is disclosed a pH-responsive pro-peptide variant of a pro-peptide of a transglutaminase wherein said pro-peptide variant is capable of modulating activity of a mature transglutaminase.

In a second embodiment, there is disclosed a pro-peptide variant that is a variant of the wild-type pro-peptide amino acid sequence set forth in SEQ ID NO: 2 comprising any of the modifications set forth in Table 2 or the sequence set forth in SEQ ID NO: 4.

In a third embodiment, there is disclosed a zymogen form of a transglutaminase comprising the pro-peptide variant of embodiments 1 or 2.

In a fourth embodiment, the mature transglutaminase, whose activity is capable of being modulated by the pro-peptide variants of embodiment 1 or 2, has antimicrobial activity.

In a fifth embodiment, there is disclosed a variant of a pro-peptide derived from a transglutaminase wherein the variant has increased binding affinity for mature transglutaminase when compared to the binding affinity of a wild-type pro-peptide of a transglutaminase.

In a sixth embodiment, there is disclosed a pro-peptide variant that is a variant of the wild-type pro-peptide amino acid sequence set forth in SEQ ID NO: 2 comprising any of the modifications set forth in Table 3 or the sequence set forth in SEQ ID NO: 4.

In a seventh embodiment, there is disclosed a zymogen form of a transglutaminase comprising the pro-peptide variant of embodiment 5 or 6.

In an eighth embodiment, the mature transglutaminase, whose activity is modulated by any of the pro-peptide variants of embodiment 5 or 6, has antimicrobial activity.

In a ninth embodiment, there is disclosed a variant of a pro-peptide derived from a transglutaminase wherein the variant has decreased binding affinity for mature transglutaminase when compared to the binding affinity of a wild-type pro-peptide of a transglutaminase.

In a tenth embodiment, there is disclosed a pro-peptide variant that is a variant of the wild-type pro-peptide amino acid sequence set forth in SEQ ID NO: 2 comprising any of the modifications set forth in Table 4.

In an eleventh embodiment, there is disclosed a zymogen form of a transglutaminase comprising the pro-peptide variant of any of embodiments 9-10.

In a twelfth embodiment, the mature transglutaminase, whose activity is modulated by any of the pro-peptide variants of any of embodiments 9-11, has antimicrobial activity.

In a thirteenth embodiment, there is disclosed a variant of a pro-peptide derived from a transglutaminase wherein the variant is a variant of the wild-type pro-peptide amino acid sequence set forth in SEQ ID NO: 2 comprising any of the modifications set forth in Table 5.

In a fourteenth embodiment, there is disclosed a zymogen form of a transglutaminase comprising the pro-peptide variant of embodiment 13.

In a fifteenth embodiment, the mature transglutaminase, whose activity is modulated by any of the pro-peptide variants of embodiment 13, has antimicrobial activity.

In a sixteenth embodiment, there is disclosed a composition comprising a zymogen form of a transglutaminase and at least one pro-peptide selected from a (i) pH-responsive pro-peptide variant of a pro-peptide of a transglutaminase, (ii) variant of a pro-peptide derived from a transglutaminase wherein the variant has increased binding affinity for mature transglutaminase when compared to the binding affinity of a wild-type pro-peptide of a transglutaminase, or (iii) variant of a pro-peptide derived from a transglutaminase wherein the variant has decreased binding affinity for mature transglutaminase when compared to the binding affinity of a wild-type pro-peptide of a transglutaminase.

In a seventeenth embodiment, there is disclosed a composition comprising a mature transglutaminase and at least one pro-peptide selected from a (i) pH-responsive pro-peptide variant of a pro-peptide of a transglutaminase, (ii) variant of a pro-peptide derived from a transglutaminase wherein the variant has increased binding affinity for mature transglutaminase when compared to the binding affinity of a wild-type pro-peptide of a transglutaminase, or (iii) variant of a pro-peptide derived from a transglutaminase wherein the variant has decreased binding affinity for mature transglutaminase when compared to the binding affinity of a wild-type pro-peptide of a transglutaminase.

In an eighteenth embodiment, the compositions of embodiment 16 or 17 comprise any the pro-peptide variants of the previous embodiments.

In a nineteenth embodiment, there is disclosed the composition of any of embodiments 16-18, wherein the mature transglutaminase has antimicrobial activity.

### BRIEF DESCRIPTION OF THE SEQUENCES

The following sequences comply with 37 C.F.R. §§ 1.831-1.835 (“Requirements for Patent Applications Containing Nucleotide Sequences and/or Amino Acid Sequence Disclosures—the Sequence Rules”) and are consistent with World Intellectual Property Organization (WIPO) Standard ST.26 (2021) and the sequence listing requirements of the European Patent Convention (EPC) and the Patent Cooperation Treaty (PCT) Rules 5.2 and 49.5 (a-bis), and Section 208 and Annex C of the Administrative Instructions. The symbols and format used for nucleotide and amino acid sequence data comply with the rules set forth in 37 C.F.R. § 1.832.

SEQ ID NO: 1 corresponds to the wild-type zymogen form of transglutaminase from Streptomyces mobaraensis. The signal sequence is in bold text and the pro-sequence is underlined.

SEQ ID NO: 2 corresponds to the wild-type pro-domain of Streptomyces mobaraensis transglutaminase having a leading methionine at the N-terminus to facilitate intracellular and/or recombinant expression.

SEQ ID NO: 3 corresponds to a thermostable variant of Streptomyces mobaraensis transglutaminase.

SEQ ID NO: 4 corresponds to a variant of the wild-type pro-domain of Streptomyces mobaraensis transglutaminase having a substitution, A24L, and additionally having a fifteen amino acid insertion between A28 and L29 (shown in bold, underlined text).

SEQ ID NO: 5 corresponds to an evolved mature form variant of Streptomyces mobaraensis transglutaminase having a hexa his-tag (bold text) and two amino acid linker (bold, underlined text).

### DETAILED DESCRIPTION

All patents, patent applications, and publications cited herein are incorporated by reference in their entireties.

Words using the singular include the plural, and vice versa, unless the context clearly dictates otherwise.

In this disclosure, many terms and abbreviations are used. The following definitions apply unless specifically stated otherwise.

As used herein, the singular forms “a,” “an,” and “the” include plural references unless the context clearly dictates otherwise. For example, the term “a compound” or “at least one compound” may include a plurality of compounds, including mixtures thereof. The terms “a,” “an,” “the,” “one or more,” and “at least one,” for example, can be used interchangeably herein.

The term “about” as used herein can allow for a degree of variability in a value or range of at most within 10%, e.g., within 5%, or within 1% of a stated value or of a stated limit of a range.

The terms “and/or” and “or” are used interchangeably herein and refer to a specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B and/or C” is intended to encompass each of the following aspects: “A, B and C”; “A, B or C”; “A or C”; “A or B”; “B or C”; “A and C”; “A and B”; “B and C”; “A” (alone); “B” (alone); and “C” (alone).

The terms “comprises,” “comprising,” “includes,” “including,” “having” and their conjugates are used interchangeably and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a methods, and those additional materials or steps that do not materially affect the basic characteristics of the material or method.

Throughout this application, various embodiments can be presented in a range format. It should be understood that the description in range format is merely for convenience and brevity and should not be construed as an inflexible limitation on the scope of the embodiments described herein. Accordingly, the description of a range should be considered to have specifically disclosed all the possible subranges as well as individual numerical values within that range. For example, description of a range, such as from 1 to 6 should be considered to have subranges such as from 1 to 2, from 1 to 3, from 1 to 4 and from 1 to 5,from 2 to 3, from 2 to 4, from 2 to 5, from 2 to 6, from 3 to 4, from 3 to 5, from 3 to 6, etc., as well as individual numbers within that range, for example, 1, 2, 3, 4, 5 and 6. This applies regardless of the breadth of the range.

“Optional” or “optionally” means that the subsequently described event, circumstance, or material may or may not occur or be present, and that the description includes instances where the event, circumstance, or material occurs or is present and instances where it does not occur or is not present.

The terms “percentage of sequence identity,” “percent identity,” “percent identical,” and “percent sequence identity” refer to comparisons between polynucleotide sequences or polypeptide sequences, and are determined by comparing two optimally aligned sequences over a comparison window, wherein the portion of the polynucleotide or polypeptide sequence in the comparison window may comprise mismatches, additions, and/or deletions (i.e., gaps) as compared to the reference sequence for optimal alignment of the two sequences. The percentage is calculated by determining the number of positions at which either the identical nucleic acid base or amino acid residue occurs in both sequences, or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (See e.g., Altschul et al., (1990) J. Mol. Biol.; 215:403-410; and Altschul et al., (1977) Nucl. Acids Res.; 3389-3402). Software for performing BLAST analyses is publicly available through the National Center for Biotechnology Information website.

One of ordinary skill in the art will appreciate that alterations in a gene at a given site which result in the production of a chemically equivalent amino acid, but do not affect the functional properties of the encoded protein, are common.

The terms “zymogen” and “proenzyme” are used interchangeably herein and refer to an inactive precursor of an enzyme, which may be converted into an active or mature enzyme by post-translational modification, for example, by catalytic action, such as via proteolytic cleavage of a pro-peptide sequence.

The terms “pro-peptide,” “pro-domain,” and “pro-region” are used interchangeably herein and refer to a N-terminal peptide leader sequence that is cleaved to afford active transglutaminase from the native zymogen form. Alternatively, this pro-peptide can be added exogenously or co-expressed as a discrete polypeptide independent of the mature transglutaminase. The pro-peptide may serve a regulatory function while the mature domain serves a catalytic function. Pro-peptides generally are recognized to have four major functions: 1) pro-peptides can function as intramolecular chaperones or folding assistants by determining the three-dimensional structure of a protein; 2) pro-peptides can function as inhibitors or activation peptides; 3) pro-peptides can direct protein sorting into specific cellular compartments or extra-cellular space and 4) pro-peptides can mediate the precursor interaction with other molecules (such as peptides, proteins, and polysaccharides) or supramolecular structures (e.g., cell walls). A single pro-peptide can perform several or even all these functions.

An “aseptilase” is an enzyme that can prevent or reduce the risk of microbial contamination through broad spectrum antimicrobial action. In other words, aseptilases act on multiple classes of microorganisms, for example Gram-positive and -negative bacteria, fungi, and viruses. Aseptilase activity may be derived from the enzyme acting directly on the cell or a cellular component, producing an antimicrobial compound or precursor as a product of catalysis, consuming one or more essential nutrients through its activity, potentiating other antimicrobials present in the environment, or any combination thereof. It has been shown that cross-linking enzymes, such as transglutaminase, can be evolved for aseptilase activity (WO2020/181099 A1, WO2021/183680 A1, WO2021/231705 A1, WO2022/055902 A1). Using directed evolution, naturally occurring enzyme activity can be evolved to have novel and/or enhanced aseptilase activity.

The terms “mature,” “active,” and “activated” are used interchangeably herein. A mature form of an enzyme, protein, polypeptide, or peptide refers to the functional form of the protein, polypeptide, or enzyme without a signal, silencing, or chaperoning pro-peptide sequence. Additionally, the mature enzyme may be truncated relative to the mature sequence while maintaining the desired activity (e.g., transglutaminase having or capable of reacting with amino acids, peptides and/or proteins).

The term “pH-responsive pro-peptide variant” refers to a pro-peptide variant whose binding affinity for mature enzyme can be modulated in response to a change in pH in its environment, for example, a change in pH producing a change in protonation state of the pro-peptide either increasing or reducing binding affinity for mature enzyme.

The terms “stability” and “thermostability” are used interchangeably to refer to increased residual activity of mature transglutaminase after incubation at elevated temperatures. In some embodiments, a pro-peptide variant increases the stability of a mature transglutaminase compared to the wild type pro-peptide. Measurement of stability is depicted in Example 4.

The term enzyme “yield” refers to the amount of protein recovered from microbial culturing. In some embodiments, a pro-peptide variant increases the yield of a mature transglutaminase compared to the wild type pro-peptide. Measurement of yield is depicted in Example 4.

The terms “signal sequence” and “signal peptide” are used interchangeably herein and refer to a sequence of amino acid residues that participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported. They can be called pre-pro-peptides or pre-pro-proteins.

The term “reversible inhibitor” refers to an inhibitor that inactivates an enzyme through noncovalent, more easily reversed, interactions. Unlike an irreversible inhibitor, a reversible inhibitor can dissociate from the enzyme. Reversible inhibitors include competitive inhibitors, noncompetitive inhibitors, and uncompetitive inhibitors.

The term “binding affinity” refers to the intermolecular interactions of a protein or enzyme to a ligand. Examples of ligands include, but are not limited to, any binding partner such as peptides, proteins, DNA, antibodies, enzymes, and some other organic molecules such as drugs. The result of binding interactions is formation of a molecular complex such as protein-protein, protein-ligand complex, and the like. For example, changes in binding affinity between mature transglutaminase and a pro-peptide are described.

The term “transglutaminase” (EC2.3.2.13) refers to a family of enzymes that catalyze the formation of an isopeptide bond between a primary amine, for example, the ε-amine of a lysine molecule, and the acyl group of a protein- or peptide-bound glutamine. Transglutaminases may catalyze a transamidation reaction between glutamyl and lysyl side chains of target proteins. Proteins possessing transglutaminase activity have been found in microorganisms, plants, and animals. transglutaminases are widely distributed in various organs, tissues, and bodily fluids. transglutaminases also form extensively crosslinked, generally insoluble, protein biopolymers that are needed for an organism to create barriers and stable structures. As used herein, a mature transglutaminase is a transglutaminase having or capable of reacting with amino acids, peptides and/or proteins.

Transglutaminases of microbial origin, unlike eukaryotic transglutaminases, are calcium-independent, which represents a major advantage for their practical use. Microbial transglutaminase (EC 2.3.2.13) is one of the most extensively studied industrial enzymes for protein functionalization and protein crosslinking because of its ability to polymerize or functionalize proteins through the formation of a stable ε-(γ-glutamyl) lysine isopeptide bond without the constraint of a consensus sequence or additional cofactors.

The most commonly used transglutaminase is transglutaminase from Streptomyces mobaraensis, the wild-type zymogen form having the amino acid sequence corresponding to SEQ ID NO: 1.

The term “amino acid” refers to the basic chemical structural unit of a protein, peptide, or polypeptide. The following abbreviations used herein to identify specific amino acids can be found in Table 1.

TABLE 1
One- and Three-Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

The terms “peptides,” “proteins,” and “polypeptides” are used interchangeably herein and refer to a polymer of amino acids joined together by peptide bonds. A “protein” or “polypeptide” comprises a polymeric sequence of amino acid residues. The single and 3-letter codes for amino acids as defined in conformity with the IUPAC-IUB Joint Commission on Biochemical Nomenclature (JCBN) are used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine(S) is represented as “G087S” or “G87S.” When describing modifications, a position followed by amino acids listed in parentheses indicates a list of modifications at that position by any of the listed amino acids. For example, 6 (L, I) means position 6 can be substituted with a leucine or isoleucine. At times, in a sequence, a slash (/) is used to define modifications, e.g., F/V, indicates that the position may have a phenylalanine or valine at that position. As a person of skill in the art would appreciate, multiple mutations at the same position cannot be used in combination (e.g., D2I cannot be used in combination with D2Q).

The term “mutation” herein refers to a change introduced into a parental sequence, including, but not limited to, modifications such as substitutions, insertions, or deletions (including truncations), thereby producing a “variant.” The consequences of a mutation include, but are not limited to, the creation of a new character, property, function, phenotype, or trait not found in the protein encoded by the parental sequence.

Related (and derivative) proteins encompass “variant,” “mutant,” or “modified” proteins, which terms are used interchangeably herein. Variant (i.e., mutant or modified) proteins differ from another (i.e., parental) protein or from one another due to modifications in one or more amino acid residues but retain at least a degree of one functional property of a parent molecule. For example, a variant may include one or more amino acid modifications such as one or more amino acid deletions/truncations, insertions, or substitutions as compared to the parental protein from which it is derived.

Alternatively or additionally, variants may have a specified degree of sequence identity with a reference protein or nucleic acid, using the BLAST percent identity algorithms. For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence and integer percentage there between. In some embodiments, the pro-peptide variant has an amino acid sequence having at least 85%, 86%, 87%, 88%, 89%, 90%, 91%, 92%, 93%, 94%, 95%, 96%, 97%, 98%, or 99% amino acid sequence identity with SEQ ID NO: 2.

The term “antimicrobial” refers to any agent or combination of agents that kills, inactivates, or inhibits the growth of any microbes such as bacteria, archaea, protozoa, fungi, algae, amoebas, viruses and the like.

The term “preservative” as used herein refers to a substance or agent that is added to a product to prevent decomposition or contamination by microbial growth, or by undesirable chemical changes. Examples of products to which preservatives may be added include, but are not limited to, food products, beverages, pharmaceutical drugs, paints, biological samples, cosmetics, wood, household cleaning products, personal care products and the like.

The terms “microorganism” and “microbe” are used interchangeably herein and refer to any living thing that is so small that it can only be seen with a microscope, i.e., a microscopic organism. Microbes may exist in a single-celled form or in a colony of cells or in a biofilm. Microbes include eukaryotes and prokaryotes such as bacteria, archaea, protozoa, fungi, algae, amoebas, viruses and the like.

As was discussed above, pro-peptides are structural elements that can determine the folding of the cognate protein, function as an inhibitor/activator peptide, mediate enzyme sorting, and mediate protein interactions with other molecules and supramolecular structures. It should be noted that a single pro-peptide can perform several or even all of these functions. Furthermore, pro-peptides make it possible to substantially alter biological properties of proteins without cardinal changes in the major functional, i.e., mature domains, of such proteins.

Disclosed herein are novel pro-peptide variants that are capable of modulating the functional activity of transglutaminase without significantly altering its mature domain. Thus, activity of the mature transglutaminase is substantially maintained.

Pro-peptide from any transglutaminase, be it from animal, plant, or microbes, may be engineered to enhance pH-responsive binding properties. Preferably, the pro-peptide is obtained from microbial transglutaminase. Most preferably, the pro-peptide is obtained from Streptomyces mobaraensis.

Pro-peptide variants were produced using directed evolution to engineer the pro-domain of microbial transglutaminase, in particular, Streptomyces mobaraensis, by using mutations to accelerate evolution, resulting in a pool of variants from which the most useful ones were selected. Directed evolution provides an alternative to rationally-designed proteins.

The pro-peptide variants disclosed in Tables 2-5 are variants of the wild-type pro-peptide amino acid sequence set forth in SEQ ID NO: 2. As was noted above, mutations can be named by the one letter code for the parent amino acid, followed by a position number and then the one letter code for the variant amino acid. For example, mutating glycine (G) at position 87 to serine(S) is represented as “G087S” or “G87S”. When describing modifications, a position followed by amino acids listed in parentheses indicates a list of modifications at that position by any of the listed amino acids. For example, 6 (L, I) means position 6 can be substituted with a leucine or isoleucine. At times, in a sequence, a slash (/) is used to define modifications, e.g., F/V, indicates that the position may have a phenylalanine or valine at that position.

TABLE 2
pH-responsive pro-peptide variants and the results
of the screenings described in Example 4 below.

| | | Improvement | |
|---|---|---|---|
| | | Improvement in pH | |
| | | responsiveness of inhibitory | |
| Position | Variant | peptide relative to WT | |
| Substitution Variants | | | |
| relative to SEQ ID NO: 2 | | | |
| 2 | D2 (I, Q, S, T) | >1.2 | fold |
| | D2P | >5 | fold |
| 7 | E7 (C, L, T) | >1.2 | fold |
| | E7 (G, N, S, V) | >5 | fold |
| 8 | E8 (C, G, L, N, R, T, Y) | >1.2 | fold |
| | E8Q | >5 | fold |
| 9 | T9 (A, G, H, K, L, Q, R, | >1.2 | fold |
| | S, V, W, Y) | | |
| 10 | K10 (D, R) | >1.2 | fold |
| | K10G | >5 | fold |
| 11 | S11 (A, D, K, L, T) | >1.2 | fold |
| 12 | Y12 (G, L, N, R, T, V) | >1.2 | fold |
| 14 | E14 (Q, R, T, V) | >1.2 | fold |
| | E14(G, K, N, S, Y) | >5 | fold |
| 15 | T15 (D, V) | >1.2 | fold |
| 18 | L18 (C, G, I, K, R, V, W) | >1.2 | fold |
| 21 | D21 (C, F, L, R, S, T, V) | >1.2 | fold |
| | D21M | >5 | fold |
| 22 | D22 (A, G, S) | >1.2 | fold |
| 24 | A24C | >1.2 | fold |
| | A24 (H, V) | >5 | fold |
| 26 | I26Y | >1.2 | fold |
| 38 | S38 (F, H, R, T, V) | >1.2 | fold |
| 46 | P46 (A, C, D, E, F, I, K, | >1.2 | fold |
| | L, M, Q, R, S, V, W) | | |
| | P46(G, N) | >5 | fold |
| Truncation Variants | | | |
| relative to SEQ ID NO: 2 | | | |
| Δ Y12-P46 | Residues 1-11 retained | >1.2 | fold |
| Δ L18-P46 | Residues 1-17 retained | >5 | fold |
| ΔI26-P46 | Residues 1-25 retained | >1.2 | fold |
| ΔS38-P46 | Residues 1-37 retained | >1.2 | fold |
| ΔP46 | Residues 1-45 retained | >1.2 | fold |

The symbol, Δ, represents a deletion of residues noted numerically relative to SEQ ID NO: 2.

TABLE 3
Variants having increased or stronger binding affinity for
mature transglutaminase when compared to the binding affinity
of a wild-type pro-peptide of a transglutaminase and the
results of the screenings described in Example 4 below.

| | | Improvement |
|---|---|---|
| | | Improvement in binding |
| | | affinity of inhibitory |
| Position | Variant | peptide relative to WT |
| Substitution Variants | | |
| relative to SEQ ID NO: 2 | | |
| 2 | D2 (F, I, P, Q, S, T) | >1.2 fold |
| 7 | E7 (C, D, H, L, N, R, S, T, V) | >1.2 fold |
| 8 | E8 (C, I, L, N, Q, R, T, V) | >1.2 fold |
| 9 | T9 (A, C, D, E, G, H, I, K, L, | >1.2 fold |
| | P, R, V, W, Y) | |
| 14 | E14 (Q, S) | >1.2 fold |
| 15 | T15 (D, Q, V) | >1.2 fold |
| 18 | L18 (F, I) | >1.2 fold |
| 21 | D21 (C, F, K, M, S, V) | >1.2 fold |
| 22 | D22 (A, H, N) | >1.2 fold |
| 24 | A24 (M, V) | >1.2 fold |
| 26 | I26V | >1.2 fold |
| 38 | S38 (F, H, T, V) | >1.2 fold |
| 46 | P46 (A, C, D, E, I, K, L, | >1.2 fold |
| | N, Q, R, S, V, W) | |
| Truncation Variants | | |
| relative to SEQ ID NO: 2 | | |
| ΔP46 | Residues 1-45 retained | >1.2 fold |

The symbol, Δ, represents a deletion of residues noted numerically relative to SEQ ID NO: 2.

TABLE 4
Variants having decreased or weaker binding affinity for mature
transglutaminase when compared to the binding affinity of
a wild-type pro-peptide of a transglutaminase and the results
of the screenings described in Example 4 below.

| | | Improvement | |
|---|---|---|---|
| | | Reduction in binding | |
| | | affinity of inhibitory | |
| Position | Variant | peptide relative to WT | |
| Substitution Variants | | | |
| relative to SEQ ID NO: 2 | | | |
| 7 | E7G | >1.2 | fold |
| 8 | E8 (G, Y) | >1.2 | fold |
| 9 | T9Q | >1.2 | fold |
| 10 | K10 (C, D, G, R) | >1.2 | fold |
| 11 | S11 (D, H, K, L, Y) | >1.2 | fold |
| | S11G | >5 | fold |
| 12 | Y12 (G, L, N, P, T, V) | >1.2 | fold |
| | Y12S | >5 | fold |
| 14 | E14 (A, C, D, G, H, N, R, T, V) | >1.2 | fold |
| 18 | L18 (E, K, S, W) | >1.2 | fold |
| 21 | D21 (L, R) | >5 | fold |
| 22 | D22 (G, P, Q, R, Y) | >1.2 | fold |
| 24 | A24 (E, H) | >1.2 | fold |
| | A24P | >5 | fold |
| 26 | I26 (A, E, Y) | >1.2 | fold |
| 38 | S38 (D, P, Y) | >1.2 | fold |
| Truncation Variants | | | |
| relative to SEQ ID NO: 2 | | | |
| ΔY12-P46 | Residues 1-11 retained | >1.2 | fold |
| ΔL18-P46 | Residues 1-17 retained | >1.2 | fold |
| ΔD22-P46 | Residues 1-21 retained | >1.2 | fold |
| ΔI26-P46 | Residues 1-25 retained | >1.2 | fold |

The symbol, Δ, represents a deletion of residues noted numerically relative to SEQ ID NO: 2.

TABLE 5
Variants having increased performance in one or more
of the following parameters as described in Example
4: Inhibition, thermostability, and yield.

| Sequence Mutations | | | | |
|---|---|---|---|---|
| (relative to SEQ ID NO. 2) | Inhibition | | Thermostability | Yield |
| D2S, E7S, D22Q | >2 | fold | — | >1.2 fold |
| D2S, E7S | >2 | fold | — | — |
| D2I, E7S, D22Q | >2 | fold | — | — |
| D2S, E7S, D21I | >1.2 | fold | — | — |
| E7S, D21T | >1.2 | fold | — | >1.2 fold |
| E7S, D21R | >1.2 | fold | — | — |
| D2I, D21T, D22Q | >1.2 | fold | — | — |
| D2F | >1.2 | fold | — | >1.2 fold |
| E7S, L18V, D21I | >2 | fold | — | — |
| L18V, D21T | >2 | fold | — | — |
| E7S, D21T, D22Q | >1.2 | fold | — | — |
| D21T, D22Q | >1.2 | fold | — | >1.2 fold |
| D2S, D21T, D22Q | >1.2 | fold | — | — |
| D2I, E7S, D21I | >1.2 | fold | — | — |
| D2S, L18V, D22Q | >2 | fold | — | — |
| D2S, D22Q | >2 | fold | — | — |
| D2I, E2S, D21T | >2 | fold | — | — |
| D2S, E7S, D21T, D22Q | >2 | fold | — | — |
| E7S, L18V, D21R | >2 | fold | — | — |
| L18V | >2 | fold | — | — |
| L18V, D21T, D22Q | >2 | fold | — | — |
| D2I, E7S, L18V, D21R | >2 | fold | — | — |
| D21T | — | | >1.2 fold | >1.2 fold |
| E7S, D22Q | >1.2 | fold | >1.2 fold | >1.2 fold |
| D2I, D22Q | >1.2 | fold | — | >1.2 fold |

The symbol, —, represents not determined, neutral, or reduced performance

Variants disclosed in Tables 2-4 were prepared using Method A of Examples 1-2. Variants disclosed in Table 5 were prepared using Method B of Examples 1-2, wherein the Pro-transglutaminase comprised the mature evolved transglutaminase sequence of SEQ ID NO: 5.

In some embodiments, there is disclosed a pH-responsive pro-peptide variant of a pro-peptide of a transglutaminase wherein said pro-peptide variant is capable of modulating activity of a mature transglutaminase.

In some embodiments, the pH-responsive pro-peptide variant is a variant of the wild-type pro-peptide amino acid sequence set forth in SEQ ID NO: 2 comprising any of the modifications set forth in Table 2 or the sequence set forth in SEQ ID NO: 4.

In some embodiments, the pro-peptide variant comprises the sequence of SEQ ID NO: 2 further comprises any of the modifications set forth in Table 2. In some embodiments, the pro-peptide variant comprises the sequence of SEQ ID NO: 2 further comprising one or more of the following substitutions: D2P, E7 (G, N, S, V), E8Q, K10G, E14 (Q, R, T, V), E14 (G, K, N, S, Y), D21M, A24 (H, V), or P46 (G, N). In some embodiments, the pro-peptide variant comprises the sequence of SEQ ID NO: 2 further comprising a deletion of L18-P46. In some embodiments, the pro-peptide variant comprises only one of the respective modifications in Table 2 and no further modifications with respect to SEQ ID NO: 2.

In some embodiments, there is disclosed a zymogen form of a transglutaminase comprising any of the pH-responsive pro-peptide variants disclosed herein. In the case of a zymogen, the pro-peptide is covalently attached to the mature domain of an enzyme, proteolytic cleavage is needed to remove the pro-peptide to produce the mature (i.e., active) enzyme. On the other hand, if the pro-peptide and the mature domain are two discrete polypeptides, the pro-peptide functions as reversible inhibitor of the mature enzyme. It is non-covalently bound to the mature domain of an enzyme thereby forming an inhibitory complex that can be disrupted. Disclosed herein are pro-peptide variants that can function as reversible inhibitors to form a pro-mature complex wherein this complex can be disrupted using a shift in pH, competition with a substrate, or modest dilution of the pro-mature complex.

The term “covalently bound” refers to a chemical bond involving the sharing of electron pairs between atoms. These electron pairs are known as shared pairs or bonding pairs, and the stable balance of attractive and repulsive forces between atoms, when they share electrons is known as covalent bonding. By way of example, the pro and mature domains may be covalently bound via a peptide bond.

The term “non-covalently bound” differs from covalently bound in that non-covalent binding does not involve the sharing of electrons but rather involves more dispersed variations of electromagnetic interactions between molecules or within a molecule. Thus, non-covalent binding of pro-peptide to mature enzyme forms either by completely exchanging electrons between atoms or by not exchanging electrons at all. Non-covalent bonds tend to be weaker than covalent bonds. Types of non-covalent bonds include but are not limited to ionic bonds, hydrogen bonds and Van der Waals interactions. The non-covalent binding of pro-peptide to mature enzyme may also occur via hydrophobic or hydrophilic interactions between the two molecules.

As was noted above, pro-peptides generally are recognized to have four major functions: 1) pro-peptides can function as intramolecular chaperones or folding assistants by determining the three-dimensional structure of a protein; 2) pro-peptides can function as inhibitors or activation peptides by maintaining the proteins (commonly enzymes) that contain them as inactive; 3) pro-peptides can direct protein sorting into specific cellular compartments or extra-cellular space; and 4) pro-peptides can mediate the precursor interaction with other molecules (such as peptides, proteins, and polysaccharides) or supramolecular structures (e.g., cell walls). A single pro-peptide can perform several or even all these functions.

In some embodiments, the mature transglutaminase of any of the embodiments described herein may additionally have antimicrobial activity.

In some embodiments, there is disclosed a variant of a pro-peptide derived from a transglutaminase wherein the variant has increased binding affinity for mature transglutaminase when compared to the binding affinity of a wild-type pro-peptide of a transglutaminase.

In some embodiments, the pro-peptide variant is a variant of the wild-type pro-peptide amino acid sequence set forth in SEQ ID NO: 2 comprising any of the modifications set forth in Table 3 or the sequence set forth in SEQ ID NO: 4. In some embodiments, the pro-peptide variant comprises the sequence of SEQ ID NO: 2 further comprising any of the modifications set forth in Table 3. In some embodiments, the pro-peptide variant comprises only one of the respective modifications in Table 4 and no further modifications with respect to SEQ ID NO: 2.

In some embodiments, the pro-peptide variant is derived from a transglutaminase pro-peptide wherein the variant has decreased binding affinity for mature transglutaminase when compared to the binding affinity of a wild-type pro-peptide of a transglutaminase.

In some embodiments, the pro-peptide variant is a variant of the wild-type pro-peptide amino acid sequence set forth in SEQ ID NO: 2 comprising any of the modifications set forth in Table 4. In some embodiments, the pro-peptide variant comprises the sequence of SEQ ID NO: 2 further comprising any of the modifications set forth in Table 4. In some embodiments, the pro-peptide variant comprises the sequence of SEQ ID NO: 2 further comprising one or more of the following substitutions: S11G, Y12S, D21 (L, R), or A24P. In some embodiments, the pro-peptide variant comprises only one of the respective modifications in Table 4 and no further modifications with respect to SEQ ID NO: 2.

In some embodiments, the pro-peptide variant causes increased inhibition of activity, thermostability, and/or yield of mature transglutaminase. In some embodiments, the pro-peptide variant comprises the sequence of SEQ ID NO: 2 further comprising any of the modifications set forth in Table 5. In some embodiments, the pro-peptide variant comprises the sequence of SEQ ID NO: 2 further comprising a combinations of substitutions selected from: D2S, E7S, and D22Q; D2S and E7s; D2I, E7S, and D22Q; E7S and D21T; E7s, L18V, and D21I; L18V and D21T; DS2, L18V, and D22Q; DS2, L18V, and D22Q; DS2 and D22Q; D2I, E2S, and D21T; D2S, E7S, D21T, and D22Q; and E7S, L18V, and D21R; L18V, D21T, and D22Q; D2I, E7S, L18V, and D21R; E7S and D22Q; and D2I and D22Q. In some embodiments, the pro-peptide variant comprises the sequence of SEQ ID NO: 2 further comprising one or more of the following substitutions: D2F and L18V. In some embodiments, the pro-peptide variant comprises the sequence of SEQ ID NO: 2 further comprising substitution of D2 with F. In some embodiments, the pro-peptide variant comprises only one of the respective modifications in Table 4 and no further modifications with respect to SEQ ID NO: 2.

It is contemplated that the disclosed pro-peptides may be in a zymogen form of a transglutaminase. In some embodiments, disclosed is a zymogen form of a transglutaminase comprising a pro-peptide variant of the disclosed embodiments.

It is contemplated that the mature transglutaminase in any of the disclosed embodiments has antimicrobial activity.

Any of the pro-peptide variants disclosed herein having stronger or weaker binding affinity for the mature transglutaminase when compared to the binding affinity of a wild-type pro-peptide of a transglutaminase, will form non-covalent bonds with the mature domain of transglutaminase with non-covalent interactions which can be disrupted in order to produce the mature (i.e., active) enzyme.

In some embodiments, the pro-peptide variant has increased binding affinity for mature transglutaminase when compared to the binding affinity of a wild-type pro-peptide of a transglutaminase. In some embodiments, the stability of a mature transglutaminase is increased in the presence of the pro-peptide variant with increased binding affinity compared to the stability of a mature transglutaminase in the presence of a wild-type pro-peptide.

In some embodiments, the pro-peptide variant has decreased binding affinity for mature transglutaminase when compared to the binding affinity of a wild-type pro-peptide of a transglutaminase. In some embodiments, the activity of the mature transglutaminase is increased in the presence of the pro-peptide variant with decreased binding affinity compared to the activity of a mature transglutaminase in the presence of a wild-type pro-peptide. In some embodiments, the yield of mature transglutaminase is increased in the presence of the pro-peptide variant with increased binding affinity compared to the yield of mature transglutaminase in the presence of a wild-type pro-peptide.

In some embodiments, the pro-peptide variant comprises the sequence of SEQ ID NO: 2 further comprising one or more modifications, and/or combinations of substitutions, in any of Tables 2-5. In some embodiments, the pro-peptide variant comprises the sequence of SEQ ID NO: 4. In some embodiments, the pro-peptide variant comprises the sequence of SEQ ID NO: 4, further comprising one or more modifications, and/or combinations of substitutions, in any of Tables 2-5. In some embodiments, the pro-peptide variant has a sequence consisting of the sequence of SEQ ID NO: 4.

In some embodiments, the disclosed pro-peptide variant of any of the disclosed embodiments may share about 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity and any integer percentage there between with the pro-peptide amino sequence set forth in SEQ ID NO: 2. In some embodiments, the pro-peptide variant comprises a sequence having at least 85%, 86%, 87%, 88%, 89%, 90%, 91%, 92%, 93%, 94%, 95%, 96%, 97%, 98%, or 99% amino acid sequence identity to SEQ ID NO: 2. In some embodiments, the pro-peptide variant comprises a sequence having at least 85%, 86%, 87%, 88%, 89%, 90%, 91%, 92%, 93%, 94%, 95%, 96%, 97%, 98%, or 99% amino acid sequence identity to SEQ ID NO: 4.

In some embodiments, the disclosed pro-peptide variant of any of the disclosed embodiments may comprise more than one amino acid substitution relative to the sequence set forth in SEQ ID NO: 2. In some embodiments, the pro-peptide variant comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids substitutions relative to the pro-peptide amino sequence set forth in SEQ ID NO: 2. In some embodiments, the pro-peptide variant comprises 15, 14, 13, 12, 11, or 10 or less amino acid substitutions relative to the pro-peptide amino acid sequence of SEQ ID NO: 2. In some embodiments, the pro-peptide variant comprises 9, 8, 7, 6, 5, or 4 or less amino acid substitutions relative to the pro-peptide amino acid sequence of SEQ ID NO: 2. In some embodiments, the pro-peptide variant comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids substitutions relative to the pro-peptide amino sequence set forth in SEQ ID NO: 4. In some embodiments, the pro-peptide variant comprises 15, 14, 13, 12, 11, or 10 or less amino acid substitutions relative to the pro-peptide amino acid sequence of SEQ ID NO: 4. In some embodiments, the pro-peptide variant comprises 9, 8, 7, 6, 5, or 4 or less amino acid substitutions relative to the pro-peptide amino acid sequence of SEQ ID NO: 4. In some embodiments, the amino acid substitutions are in consecutive positions in the pro-peptide amino sequence. In some embodiments, the amino acid substitutions are in non-consecutive positions in the pro-peptide amino sequence.

In some embodiments, the disclosed the pro-peptide variant of any of the disclosed embodiments may comprise at least one amino acid deletion relative to the pro-peptide amino sequence set forth in SEQ ID NO: 2. In some embodiments, the pro-peptide variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids deletions of the pro-peptide amino acid sequence set forth in SEQ ID NO: 2. In some embodiments, the pro-peptide variant comprises no more than 10, 9, 8, 7, 6, 5, 4, 3, 2, or 1 amino acid deletions of the amino acid sequence of SEQ ID NO: 2, such as no more than 5, 4, 3, 2, or 1 amino acid deletions. In some embodiments, the pro-peptide variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids deletions of the pro-peptide amino acid sequence set forth in SEQ ID NO: 4. In some embodiments, the pro-peptide variant comprises no more than 10, 9, 8, 7, 6, 5, 4, 3, 2, or 1 amino acid deletions of the amino acid sequence of SEQ ID NO: 4, such as no more than 5, 4, 3, 2, or 1 amino acid deletions. In some embodiments, the amino acid deletions are in consecutive positions in the pro-peptide amino sequence. In some embodiments, the amino acid deletions are in non-consecutive positions in the pro-peptide amino sequence.

In some embodiments, the disclosed pro-peptide variant of any of the disclosed embodiments may comprise at least one truncation compared to the pro-peptide amino sequence set forth in SEQ ID NO: 2. In some embodiments, the truncation comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids of the pro-peptide amino sequence set forth in SEQ ID NO: 2. In some embodiments, the truncation comprises no more than 35 amino acids, no more than 30 amino acids, no more than 25 amino acids, no more than 20 amino acids, no more than 15 amino acids, no more than 10 amino acids, or no more than 5 amino acids of the sequence of SEQ ID NO: 2. In some embodiments, the truncation is no more than 1 amino acid of the sequence of SEQ ID NO: 2. In some embodiments, the truncation comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids of the pro-peptide amino sequence set forth in SEQ ID NO: 4. In some embodiments, the truncation comprises no more than 35 amino acids, no more than 30 amino acids, no more than 25 amino acids, no more than 20 amino acids, no more than 15 amino acids, no more than 10 amino acids, or no more than 5 amino acids of the sequence of SEQ ID NO: 2. In some embodiments, the truncation is no more than 1 amino acid of the sequence of SEQ ID NO: 4. In some embodiments, the truncation occurs at the N-terminal end of the pro-peptide amino sequence. In some embodiments, the truncation occurs at the C-terminal end of the pro-peptide amino sequence.

In some embodiments, the disclosed pro-peptide variant of any of the disclosed embodiments may comprise at least one insertion compared to the pro-peptide amino acid sequence set forth in SEQ ID NO: 2. In some embodiments, the insertion comprises 1, 2, 3, 4, 5, 6, 7, 8, 9, 10, 11, 12, 13, 14, 15, 16, 17, 18, 19, or 20 amino acids. In some embodiments, the insertion comprises no more than 5 amino acids, no more than 10 amino acids, or no more than 15 amino acids. In some embodiments, the pro-peptide variant comprises the sequence of SEQ ID NO: 2 and further comprises the 15 amino acid insertion disclosed in SEQ ID NO: 4.

### Compositions

Also encompassed herein are compositions comprising a combination of a zymogen form of a transglutaminase or a mature transglutaminase and one or more of a (i) pH-responsive pro-peptide variant of a pro-peptide of a transglutaminase, (ii) variant of a pro-peptide derived from a transglutaminase wherein the variant has increased binding affinity for mature transglutaminase when compared to the binding affinity of a wild-type pro-peptide of a transglutaminase, or (iii) variant of a pro-peptide derived from a transglutaminase wherein the variant has decreased binding affinity for mature transglutaminase when compared to the binding affinity of a wild-type pro-peptide of a transglutaminase.

In some cases, only a single pro-peptide variant may be needed, if that pH-responsive pro-peptide variant or pro-peptide variant is able perform the necessary functions, for example, as an intramolecular chaperone (or folding assistant) and as an inhibitory or activating peptide.

In addition, as was noted above, the mature transglutaminase may have antimicrobial activity.

In some embodiments, there is a composition comprising a zymogen form of a transglutaminase and at least one pro-peptide selected from (i) a pH-responsive pro-peptide variant of a pro-peptide of a transglutaminase, (ii) variant of a pro-peptide derived from a transglutaminase wherein the variant has increased binding affinity for mature transglutaminase when compared to the binding affinity of a wild-type pro-peptide of a transglutaminase, or (iii) variant of a pro-peptide derived from a transglutaminase wherein the variant has decreased binding affinity for mature transglutaminase when compared to the binding affinity of a wild-type pro-peptide of a transglutaminase.

In some embodiments, there is disclosed a composition comprising a mature transglutaminase and at least one pro-peptide selected from (i) a pH-responsive pro-peptide variant of a pro-peptide of a transglutaminase, (ii) variant of a pro-peptide derived from a transglutaminase wherein the variant has increased binding affinity for mature transglutaminase when compared to the binding affinity of a wild-type pro-peptide of a transglutaminase, or (iii) variant of a pro-peptide derived from a transglutaminase wherein the variant has decreased binding affinity for mature transglutaminase when compared to the binding affinity of a wild-type pro-peptide of a transglutaminase.

Furthermore, any of the compositions disclosed herein may comprise any of the pH-responsive pro-peptide variant or pro-peptide variants disclosed herein. Specifically, (i) the pH responsive pro-peptide variant is a variant of the wild-type pro-peptide amino acid sequence set forth in SEQ ID NO: 2 comprising any of the modifications set forth in Table 2 or the sequence set forth in SEQ ID NO: 4; (ii) the pro-peptide variant is a variant of the wild-type pro-peptide amino acid sequence set forth in SEQ ID NO: 2 comprising any of the modifications set forth in Table 3 or the sequence set forth in SEQ ID NO: 4; or (iii) the pro-peptide variant is a variant of the wild-type pro-peptide amino acid sequence set forth in SEQ ID NO: 2 comprising any of the modifications set forth in Table 4.

Non-limiting embodiments of the foregoing disclosed herein include:
1. A pH-responsive pro-peptide variant of a pro-peptide of a transglutaminase wherein said pro-peptide variant is capable of modulating activity of a mature transglutaminase.
2. A pro-peptide variant that is a variant of the wild-type pro-peptide amino acid sequence set forth in SEQ ID NO: 2 comprising any of the modifications set forth in Table 2 or the sequence set forth in SEQ ID NO: 4.
3. A pro-peptide variant of embodiment 1 or 2, wherein the variant shares about 50%, 55%, 60%, 65%, 70%, 75%, 80%, 85%, 86%, 87%, 88%, 89%, 90%, 91%, 92%, 93%, 94%, 95%, 96%, 97%, 98%, or 99% amino acid sequence identity with the sequence set forth in SEQ ID NO: 2.
4. A pro-peptide variant of embodiment 1 or 2, wherein the variant comprises 5 or less amino acid substitutions with respect to SEQ ID NO: 2.
5. The pro-peptide variant of embodiment 1 or 2, wherein the pro-peptide variant comprises a deletion selected from: deletion of Y12-P46; deletion of L18-P46; deletion of 126-P46; deletion of S38-P46; or deletion of P46, wherein the pro-peptide comprises no further modifications with respect to SEQ ID NO: 2.
6. A zymogen form of a transglutaminase comprising the pH-responsive pro-peptide of any one of embodiments 1-5.
7. The pro-peptide of a transglutaminase of any one of embodiments 1-5, wherein the mature transglutaminase has antimicrobial activity.
8. A variant of a pro-peptide derived from a transglutaminase wherein the variant has increased binding affinity for mature transglutaminase when compared to the binding affinity of a wild-type pro-peptide of a transglutaminase.
9. A pro-peptide variant that is a variant of the wild-type pro-peptide amino acid sequence set forth in SEQ ID NO: 2 comprising any of the modifications set forth in Table 3 or the sequence set forth in SEQ ID NO: 4.
10. The pro-peptide variant of embodiment 7 or 8, wherein the variant shares about 50%, 55%, 60%, 65%, 70%, 75%, 80%, 85%, 86%, 87%, 88%, 89%, 90%, 91%, 92%, 93%, 94%, 95%, 96%, 97%, 98%, or 99% amino acid sequence identity with the sequence set forth in SEQ ID NO: 2.
11. The pro-peptide variant of embodiment 8 or 9, wherein the variant comprises 5 or less amino acid substitutions with respect to SEQ ID NO: 2.
12. The pro-peptide variant of any one of embodiments 8-11, wherein the stability of a mature transglutaminase is increased in the presence of the pro-peptide variant when compared to the stability of a mature transglutaminase in the presence of a wild-type pro-peptide.
13. The pro-peptide variant of any one of embodiments 8-11, where in the yield of a mature transglutaminase is increased in the presence of the pro-peptide variant when compared to the stability of a mature transglutaminase in the presence of a wild-type pro-peptide.
14. A zymogen form of a transglutaminase comprising the pro-peptide variant of any one of embodiments 8-13.
15. The pro-peptide variant of any one of embodiments 8-13, wherein the mature transglutaminase has antimicrobial activity.
16. A variant of a pro-peptide derived from a transglutaminase wherein the variant has decreased binding affinity for mature transglutaminase when compared to the binding affinity of a wild-type pro-peptide of a transglutaminase.
17. A pro-peptide variant that is a variant of the wild-type pro-peptide amino acid sequence set forth in SEQ ID NO: 2 comprising any of the modifications set forth in Table 4.
18. The pro-peptide variant of embodiment 16 or 17, wherein the variant shares about 50%, 55%, 60%, 65%, 70%, 75%, 80%, 85%, 86%, 87%, 88%, 89%, 90%, 91%, 92%, 93%, 94%, 95%, 96%, 97%, 98%, or 99% amino acid sequence identity with the sequence set forth in SEQ ID NO: 2.
19. The pro-peptide variant of embodiment 16 or 17, wherein the variant comprises 5 or less amino acid substitutions with respect to SEQ ID NO: 2
20. The pro-peptide variant of embodiment 16 or 17, wherein the variant pro-peptide comprises a deletion selected from: deletion of Y12-P46; deletion of L18-P46; deletion of D22-P46; or deletion of 126-P46, wherein the pro-peptide comprises no further modifications with respect to SEQ ID NO: 2
21. The pro-peptide variant of any one of embodiments 16-20, wherein the activity of a mature transglutaminase activity is increased in the presence of the pro-peptide variant compared to the activity of a mature transglutaminase in the presence of a wild-type pro-peptide.
22. A zymogen form of a transglutaminase comprising the pro-peptide variant of any one of embodiments 16-21.
23. The pro-peptide variant of any one of embodiments 16-21, wherein the mature transglutaminase has antimicrobial activity.
24. A pro-peptide derived from a transglutaminase wherein the variant is a variant of the wild-type pro-peptide amino acid sequence set forth in SEQ ID NO: 2 comprising any of the modifications set forth in Table 5.
25. The pro-peptide variant of embodiment 24, wherein the variant shares about 50%, 55%, 60%, 65%, 70%, 75%, 80%, 85%, 86%, 87%, 88%, 89%, 90%, 91%, 92%, 93%, 94%, 95%, 96%, 97%, 98%, or 99% amino acid sequence identity with the sequence set forth in SEQ ID NO: 2.
26. The pro-peptide variant of embodiment 24, wherein the variant comprises 5 or less amino acid substitutions with respect to SEQ ID NO: 2
27. A zymogen form of a transglutaminase comprising the pro-peptide variant of any one of embodiments 24-26.
28. The pro-peptide variant of any one of embodiments 24-26, wherein the mature transglutaminase has antimicrobial activity.
29. The pro-peptide variant of any of the prior embodiments wherein the pro-peptide variant has an N-terminal methionine.
30. A composition comprising a zymogen form of a transglutaminase and at least one pro-peptide selected from (i) a pH-responsive pro-peptide variant of a pro-peptide of a transglutaminase, (ii) variant of a pro-peptide derived from a transglutaminase wherein the variant has increased binding affinity for mature transglutaminase when compared to the binding affinity of a wild-type pro-peptide of a transglutaminase, or (iii) variant of a pro-peptide derived from a transglutaminase wherein the variant has decreased binding affinity for mature transglutaminase when compared to the binding affinity of a wild-type pro-peptide of a transglutaminase.
31. A composition comprising a mature transglutaminase and at least one pro-peptide selected from a (i) pH-responsive pro-peptide variant of a pro-peptide of a transglutaminase, (ii) variant of a pro-peptide derived from a transglutaminase wherein the variant has increased binding affinity for mature transglutaminase when compared to the binding affinity of a wild-type pro-peptide of a transglutaminase, or (iii) variant of a pro-peptide derived from a transglutaminase wherein the variant has decreased binding affinity for mature transglutaminase when compared to the binding affinity of a wild-type pro-peptide of a transglutaminase.
32. The composition of embodiment 30 or 31 wherein the mature transglutaminase has antimicrobial activity.
33. The composition of any one of embodiments 30-32 wherein the (i) pH-responsive pro-peptide variant is a variant of the wild-type pro-peptide amino acid sequence set forth in SEQ ID NO: 2 comprising any of the modifications set forth in Table 2 or the sequence set forth in SEQ ID NO: 4.
34. The composition of any one of embodiments 30-32 wherein the (ii) pro-peptide variant is a variant of the wild-type pro-peptide amino acid sequence set forth in SEQ ID NO: 2 comprising any of the modifications set forth in Table 3 or the sequence set forth in SEQ ID NO: 4.
35. The composition of any one of embodiments 30-32 wherein the (iii) pro-peptide variant is a variant of the wild-type pro-peptide amino acid sequence set forth in SEQ ID NO: 2 comprising any of the modifications set forth in Table 4.
36. The composition of any one of embodiments 30-32 wherein the (ii) pro-peptide variant or (iii) pro-peptide variant is a variant of the wild-type pro-peptide amino acid sequence set forth in SEQ ID NO: 2 comprising any of the modifications set forth in Table 5.
37. A composition comprising a zymogen form of a transglutaminase and at least one pro-peptide of any one of embodiments 1-29.
38. A composition comprising a mature transglutaminase and at least one pro-peptide of any one of embodiments 1-29.

### EXAMPLES

The following examples are intended to illustrate, but not limit, the invention. Accordingly, from the above discussion and the Examples, one skilled in the art can ascertain essential characteristics of this disclosure, and without departing from the spirit and scope thereof, can make various changes and modifications to adapt to various uses and conditions.

Unless defined otherwise herein, all technical and scientific terms used herein have the same meaning as commonly understood by one of ordinary skill in the art to which this disclosure belongs.

### Example 1

### Creation of Pro-Peptide Variants

Method A. A bicistronic expression vector encoding the wild-type pro-domain of transglutaminase (SEQ ID NO: 2) and a thermostable mature transglutaminase variant (SEQ ID NO: 3) as separate polypeptides was cloned as described in PCT/US20/49226 (WO2021/178001 A1). The vector was used as a template for synthesis of a random mutagenesis library targeting the pro-domain coding sequence to create pro-domain peptide variants.

Method B. The gene coding for a Pro-transglutaminase (i.e., zymogen form of transglutaminase) variant with the wild-type pro-domain (SEQ ID NO: 2) and an evolved mature domain including a C-terminal His tag (SEQ ID NO: 5), and was cloned into a pET vector operatively linked to the T7 promoter. The expression vector also contains the pMB1 origin of replication and a kanamycin resistance gene. The resulting plasmid was transformed first into E. coli DH-10B, using standard methods known in the art. The transformants were isolated by subjecting the cells to kanamycin selection, as known in the art (See, e.g., U.S. Pat. No. 8,383,346 and WO2010/144103, both of which are incorporated by reference herein, in their entirety), and the sequence of the Pro-transglutaminase gene was verified by Sanger sequencing. The plasmid was recovered from a positive clone, using methods known in the art, and transformed into E. coli BL21 (DE3) for expression. Mutations were subsequently introduced into the pro-region of the Pro-transglutaminase gene using a combination of de novo DNA synthesis as described in PCT/US20/49226 and site directed mutagenesis methods known in the art.

### Example 2

### Expression of Pro-Peptide Variants in Multiwell Plates

Method A. Plasmid libraries containing mature transglutaminase and variant pro-domain peptides were plated on Luria-Bertani (LB) agar plates containing 50 μg/mL of kanamycin. After incubation for at least 16 hours at 37° C., colonies were picked and inoculated into 1 mL of LB broth with 50 μg/mL kanamycin in one well of a 96 deep well culture plate. The plate was covered with a breathable seal and incubated overnight at 37° C. with 400 rpm of shaking. 10 μL of the seed culture was used to inoculate 1 mL of media in a 96 deep well culture plate for expression. The expression plate was allowed to grow for 6-8 hours at 30° C. Expression was then induced by adding isopropyl beta-D-1-thiogalactopyranoside (IPTG) to a final concentration of 1 mM and the temperature was reduced to 20° C. while growth continued for an additional 15 hours. Cells were harvested by centrifugation at 4000 rcf for 30 minutes and pellets were frozen at −80° C.

Method B. The E. coli strain BL21 (DE3), containing the Pro-transglutaminase expression vector, was cultured overnight in Luria broth at 37° C. until the culture reached saturation. The following morning, the culture was used to inoculate wells of a 96-deep well plate containing a medium including glycerol, soy peptone, yeast extract, magnesium sulfate heptahydrate, potassium phosphate monobasic, and 50 μg/mL Kanamycin at 30-34° C. for up to 10 hours with continuous shaking. Isopropyl β-d-1-thiogalactopyranoside (IPTG) was added to a final concentration of 0.1-1 mM and incubation was continued at 18-25° C. for up to 24 hours.

### Example 3

### Activation of Pro-Transglutaminase Variants

Soluble transglutaminase-activating M4 metalloprotease (TAMEP) was added at 1-5% vol to Pro-transglutaminase variant clarified lysate. The reactions were then incubated at 37° C. After incubating with soluble TAMEP for 15-120 minutes, soluble Streptomyces mobaraensis transglutaminase-activating tripeptidyl aminopeptidase (SM-TAP) was added to the reaction at 1-5% vol. The reactions were then incubated at 37° C. for an additional 15-120 minutes.

### Example 4

### Screening of Pro-Peptide Variants

The pro-domain peptide variants were expressed as described in Example 2, with mature transglutaminase activated as described in Example 3 if expressed in zymogen form, and assayed using a primary screen, i.e., the standard colorimetric hydroxamate activity assay for transglutaminase (Folk and Cole (1965) J Biol Chemistry 240 (7): 2951-60). Briefly, the hydroxamate assay uses N-benzyloxycarbonyl-L-glutaminyl-glycine (ZQG) as a low molecular weight amine acceptor substrate and hydroxylamine as an amine donor. In the presence of transglutaminase, the hydroxylamine is incorporated to form Z-glutamylhydroxamate-glycine, which develops a colored complex with iron (III), detectable at 525 nm after incubation at 37° C. for 5-60 minutes. The calibration was performed using L-glutamic acid γ-monohydroxamate (Millipore Sigma) as standard. One unit of transglutaminase is defined as the amount of enzyme that catalyzes formation of 1 μmol of the peptide derivative of γ-glutamylhydroxylamine per minute. The expression level of transglutaminase in E. coli lysate was measured using a commercially available ELISA (Zedira E021). An alternative method for protein quantification of transglutaminase in lysate was employed that used the commercially available bicinchoninic acid protein assay kit (ThermoFisher 23225).

A secondary screen was performed to determine initial rate of transglutaminase in the presence of the pro-peptide variants using transglutaminase-catalyzed labeling of casein with dansylcadaverine (e.g., a commercially available kit such as the Transglutaminase Fluorogenic Activity Assay Kit, T036, Zedira, Germany). Transglutaminase activity was monitored by measuring the fluorescence (excitation wavelength 332 nm; emission wavelength 500 nm) using a BioTek Synergy H1 microplate reader. Transglutaminase-catalyzed covalent coupling (crosslinking) of monodansylcadaverine with N,N-dimethylcasein produces a product that causes a shift in intensity and wavelength of fluorescence of the dansyl group now linked to the casein. The relative transglutaminase activity is shown by increase of fluorescence intensity over time. The data were normalized for transglutaminase expression levels in E. coli lysate as described above. Pro-domain variants were identified based on their ability to alter the activity of the mature transglutaminase enzyme.

Two activity assays were performed as described above. Wherein, the mature enzyme was assayed in the presence of the pro-peptide variants.

To evaluate pH-responsiveness, initial rates of the active transglutaminase in the presence of pro-peptide variants were determined at pH 6.0 and pH 8.0 using the casein assay. Briefly, this assay was conducted at pH 6.0 and pH 8.0 for each pro-peptide variant to interrogate pH-responsive modulation of mature transglutaminase activity. Activity of the mature enzyme in the presence of each pro-peptide variant was normalized against transglutaminase expression levels using a commercially available ELISA (Zedira E021).

Pro-peptide variants created and expressed according to Method A in Examples 1 and 2 above that demonstrated an increase in activity at pH 6.0 relative to pH 8.0 compared to the wild-type pro-peptide are disclosed in Table 2 and SEQ ID NO: 4.

Pro-peptide variants created and expressed according to Method A in Examples 1 and 2 above that demonstrated a decrease in transglutaminase activity relative to the wild-type pro-peptide, using the casein assay, are disclosed in Table 3 and SEQ ID NO: 4. It is believed that these pro-peptide variants have increased binding affinity with mature transglutaminase, which leads to increased stabilization of mature transglutaminase.

Pro-peptide variants created and expressed according to Method A in Examples 1 and 2 above that demonstrated an increase in transglutaminase activity relative to the wild-type pro-peptide, using the casein assay, are disclosed in Table 4. It is believed that these pro-peptide variants have reduced binding affinity with mature mTgase.

Pro-peptide variants created and expressed according to Method B in Examples 1 and 2 above that demonstrated a decrease in transglutaminase activity relative to the wild-type pro-peptide, using the casein assay, are disclosed in Table 5. The Pro-transglutaminase used to create these variants comprised the mature evolved transglutaminase sequence of SEQ ID NO: 5. The antimicrobial activity of the mature evolved transglutaminase (SEQ ID NO: 5) is demonstrated in Example 5. It is believed that these pro-peptide variants have increased binding affinity with mature transglutaminase. To evaluate inhibition of variants in Table 5, activated pro-peptide variants expressed as described in Example 2, method B, and activated as described in Example 3, were diluted approximately 20-fold into the casein assay solution and assayed as described above.

Pro-peptide variants that demonstrated an increase in transglutaminase thermostability relative to the wild-type pro-peptide are disclosed in Table 5. These pro-peptide variants demonstrated increased transglutaminase activity after being held at between 37° C.-50° C. for 30 minutes and diluted to between 10-fold to 100-fold. It is believed that these pro-peptide variants confer thermal stability to mature transglutaminase.

Pro-peptide variants that demonstrated an increase in transglutaminase yield relative to the wild-type pro-peptide are disclosed in Table 5. These pro-peptide variants demonstrated increased zymogen transglutaminase yield compared to the wild-type pro-domain as measured by bicinchoninic acid assay.

### Example 5

### Characterization of an Evolved Transglutaminase

The E. coli strain BL21 (DE3), containing the Pro-transglutaminase expression vector described in Example 1B, was cultured overnight in Luria broth at 37° C. until the culture reached saturation. The following morning, the culture was used to inoculate a shake flask containing a medium including glycerol, soy peptone, yeast extract, magnesium sulfate heptahydrate, and potassium phosphate monobasic, at 30-34° C. for up to 10 hours with continuous shaking. Isopropyl β-d-1-thiogalactopyranoside (IPTG) was added to a final concentration of 0.1-1 mM and incubation was continued at 20-25° C. for up to 24 hours.

Cells were harvested by centrifugation at 8000×g for up to 60 minutes. The supernatant was discarded, and the pellet was resuspended to 20% w/v in 50 mM phosphate buffer, pH 7.4. The cells were lysed using a high-pressure homogenizer at pressures from 15000-20000 psi. The crude lysates were clarified through centrifugation at 15000×g for up to 60 minutes. The clarified lysate containing Pro-transglutaminase was assessed by sodium dodecyl sulfate polyacrylamide gel electrophoresis (SDS-PAGE) and by spectroscopy. After confirming the presence of Pro-transglutaminase, in the clarified lysate, the sample was buffer exchanged against a solution of 50 mM phosphate buffer pH 7.4, 100 mM sodium chloride, 10 mM ammonium sulfate, and 20% glycerol on a 10- to 50 kDa membrane.

Alternatively, the Pro-transglutaminase variant may be secreted, for example, from a microbial strain that is known to those skilled in the art to secrete transglutaminase such as Streptomyces mobaraensis or Bacillus subtilis. The pellet is discarded and the supernatant is recovered and is assessed by SDS-PAGE and by spectroscopy. After confirming the presence of Pro-transglutaminase in the supernatant, the sample is buffer exchanged against a solution of 50 mM phosphate buffer pH 7.4, 100 mM sodium chloride, 10 mM ammonium sulfate, and 20% glycerol on a 10- to 50 kDa membrane.

Following buffer exchange, soluble TAMEP was added at 1% vol to purified Pro-transglutaminase variant. The reactions were then incubated at 37° C., 300 rpm. After incubating with soluble TAMEP for 15-120 minutes, soluble SM-TAP was added to the reaction at 1% vol. The reactions were then incubated at 37° C., 300 rpm for an additional 15-120 minutes Following activation, ethylenediaminetetraacetic acid (EDTA) and glycerol were added to a final concentration of 1 mM and 30%, respectively, and transglutaminase activity was assessed using the Colorimetric Activity Assay described in Example 4.

Purified mature transglutaminase variants were stored at a concentration of 10 g/L in 30% glycerol, 50 mM phosphate buffer pH 7.4, 100 mM sodium chloride, 10 mM ammonium sulfate, and 1 mM EDTA.

E. coli ATCC 8739 and C. albicans ATCC 10231 were acquired from the American Type Culture Collection (ATCC) (Manassas, VA) and maintained as −80° C. frozen glycerol stocks.

For Minimum Inhibitor Concentration (MIC) determination of bacterial cultures, E. coli ATCC 8739 was grown overnight (16-18 hours) in Mueller Hinton broth at 37° C. The following day, the cell density of the saturated cultures was calculated using OD₆₀₀ and cultures were diluted to 104 to 106 CFU/mL in sterile Mueller Hinton broth to generate the inoculum, and 100 μL of the inoculum was combined with 100 μL of serially diluted transglutaminase variant at a range of 0.0001-0.05 weight percent. Growth curves were measured by OD₆₀₀ on a BioTek® Synergy Plate Reader. All test conditions were performed in triplicate. MIC was determined to be 50 ppm (0.005% w/v) against E. coli for the enzyme described in SEQ ID NO: 5 using OD₆₀₀. MIC was outside the range of detection against E. coli for the enzyme described in SEQ ID NO: 3 using OD₆₀₀ (as described in Example 3).

For MIC determination of yeast cultures, C. albicans ATCC 10231 was grown overnight (24 hours) in Sabouraud Dextrose broth at 30° C. The following day, the cell density of the saturated cultures was calculated using OD₆₀₀ and cultures were diluted to 104 to 106 CFU/mL in sterile Sabouraud Dextrose broth media to generate the inoculum, and 100 μL of the inoculum was combined with 100 μL of serially diluted transglutaminase variant at a range of 0.0001-0.05 weight percent. The cultures were grown overnight at 30° C. and growth curves were measured by OD₆₀₀ on a BioTek® Synergy Plate Reader. All test conditions were performed in triplicate. MIC was determined to be 50 ppm (0.005% w/v) against C. albicans for the enzyme described in SEQ ID NO: 5 using OD₆₀₀. MIC was determined to be 25 ppm (0.0025% w/v) against C. albicans for the enzyme described in SEQ ID NO: 3 using OD₆₀₀ (as described in Example 3).

SEQUENCE LISTING
Wild-type Streptomyces mobaraensis
transglutaminase zymogen (Pre-Pro-
transglutaminase; Uniprot P81453) with
its signal sequence in bold and pro-domain
underlined
SEQ ID NO: 1
MRIRRRALVFATMSAVLCTAGFMPSAGEAAADNGAGEETKSYAET
YRLTADDVANINALNESAPAASSAGPSFRAPDSDDRVTPPAEPLD
RMPDPYRPSYGRAETVVNNYIRKWQQVYSHRDGRKQQMTEEQREW
LSYGCVGVTWVNSGQYPTNRLAFASFDEDRFKNELKNGRPRSGET
RAEFEGRVAKESFDEEKGFQRAREVASVMNRALENAHDESAYLDN
LKKELANGNDALRNEDARSPFYSALRNTPSFKERNGGNHDPSRMK
AVIYSKHFWSGQDRSSSADKRKYGDPDAFRPAPGTGLVDMSRDRN
IPRSPTSPGEGFVNFDYGWFGAQTEADADKTVWTHGNHYHAPNGS
LGAMHVYESKFRNWSEGYSDFDRGAYVITFIPKSWNTAPDKVKQG
WP
Wild-type Streptomyces mobaraensis
transglutaminase pro-domain having a
leading methionine at the N-terminus
to facilitate intracellular and/or
recombinant expression
SEQ ID NO: 2
MDNGAGEETKSYAETYRLTADDVANINALNESAPAASSAGPSFRAP
A thermostable variant of Streptomyces
mobaraensis transglutaminase
SEQ ID NO: 3
MDPDDRVTPPAEPLDRMPDPYRPSYGRAETVVNNYIRKWQQVYSH
RDGRKQQMTEEQREWLSYGCVGVTWVNSGQYPTNRLAFASFDEDR
FKNELKNGRPRSGETRAEFEGRVAKESFDEEKGFQRAREVASVMN
RALENAHDESAYLDNLKKELANGNDALRNEDARSPFYSALRNTPS
FKERNGGNHDPSRMKAVIYSKHFWSGQDRSSSADKRKYGDPDAFR
PAPGTGLVDMSRDRNIPRSPTSPGEGFVNFDYGWFGAQTEADADK
TVWTHGNHYHAPNGSLGAMHVYESKFRNWSEGYSDFDRGAYVITF
IPKSWNTAPDKVKQGWP
A variant of the wild-type pro-domain
of Streptomyces mobaraensis transglutaminase
having a substitution, A24L, and additionally
having a fifteen amino acid insertion
between A28 and L29
SEQ ID NO: 4
MDNGAGEETKSYAETYRLTADDVLNINAETYRLTADDVRNINALN
ESAPAASSAGPSFRAP
An evolved variant of mature Streptomyces
mobaraensis transglutaminase, including a
two amino acid linker and hexa-his tag
SEQ ID NO: 5
MDPDDRVTPPAEPLDRMPDPYRPSYGRAETVVNNYIRKWQQVYSH
RDGRKSQMTEEQREWLSYGCVGVTWVNSGAYPTNRLAFASFDEDR
FHNELKNGRPRSGETRAEFEGRVAKESFDEEKGFLRAREVASVMN
RALENAHDESAYLDNLRRELANGNDALRNEDARSPFYSALRNTPS
FKERNGGNHDPSRMKAVIYSKHFWSGQDRSSSADKRKYGDPDAFR
PAPGTGLVDMSRDRNIPRSPTSPGEGFVNFDYGWFGAQTEADADK
TVWTHGNHYHAPMASLGAMHVYESKFRNWSEGYSDFDRGAYVITF
IPKSWNTAPDKVKQGWPLEHHHHHH

1. A pro-peptide variant of a transglutaminase pro-peptide, wherein the pro-peptide variant comprises:
(i) the sequence of SEQ ID NO: 2 further comprising one or more modifications comprising one or more of the following:
substitution of D2 with I, P, Q, S, or T;
substitution of E7 with C, L, G, N, S, T, or V;
substitution of E8 with C, G, L, N, Q, R, T, or Y;
substitution of T9 with A, G, H, K, L, Q, R, S, V, W, or Y;
substitution of K10 with D, G, R;
substitution of S11 with A, D, K, L, or T;
substitution of Y12 with G, L, N, R, T, or V;
substitution of E14 with G, K, N, Q, R, S, T, V, or Y;
substitution of T15 with D or V;
substitution of L18 with C, G, I, K, R, V, or W;
substitution of D21 with C, F, L, M, R, S, T, or V;
substitution of D22 with A, G, or S;
substitution of A24 with C, H, or V;
substitution of 126 with Y
substitution of S38 with F, H, R, T, or V;
substitution of P46 with A, C, D, E, F, G, I, K, L, M, N, Q, R, S, V, or W;
deletion of Y12-P46;
deletion of L18-P46;
deletion of 126-P46;
deletion of S38-P46; or
deletion of P46; or
(ii) the sequence of SEQ ID NO: 4.

2. The pro-peptide variant of claim 1, wherein the pro-peptide variant comprises a sequence having at least 85% sequence identity to SEQ ID NO: 2.

3. The pro-peptide variant of claim 1, wherein the pro-peptide variant comprises 5 or less amino acid substitutions with respect to SEQ ID NO: 2.

4. The pro-peptide variant of claim 1, wherein the pro-peptide variant comprises a deletion selected from:
deletion of Y12-P46;
deletion of L18-P46;
deletion of I26-P46;
deletion of S38-P46; and
deletion of P46,
wherein the pro-peptide comprises no further modifications with respect to SEQ ID NO: 2.

5. A zymogen form of a transglutaminase comprising the pro-peptide variant of claim 1.

6. The pro-peptide variant of claim 1, wherein the mature transglutaminase has antimicrobial activity.

7. A pro-peptide variant of a transglutaminase pro-peptide, wherein the pro-peptide variant comprises:
(i) the sequence of SEQ ID NO: 2 further comprising one or more modifications comprising one or more of the following:
substitution of D2 with F, I, P, Q, S, or T;
substitution of E7 with C, D, H, L, N, R, S, T, or V;
substitution of E8 with C, I, L, N, Q, R, T, or V;
substitution of T9 with A, C, D, E, G, H, I, K, L, P, R, V, W, or Y;
substitution of E14 with Q or S;
substitution of T15 with D, Q, or V;
substitution of L18 with F or I;
substitution of D21 with C, F, K, M, S, or V;
substitution of D22 with A, H, or N;
substitution of A24 with M or V;
substitution of 126 with V;
substitution of S38 with F, H, T, or V;
substitution of P46 with A, C, D, E, I, K, L, N, Q, R, S, V, or W; or
deletion of P46; or
(ii) the sequence of SEQ ID NO: 4.

8. The pro-peptide variant of claim 7, wherein the pro-peptide variant comprises a sequence having at least 85% sequence identity to SEQ ID NO: 2.

9. The pro-peptide variant of claim 7, wherein the pro-peptide variant comprises 5 or less amino acid substitutions with respect to SEQ ID NO: 2.

10. A zymogen form of a transglutaminase comprising the pro-peptide variant of claim 7.

11. The pro-peptide variant of claim 7, wherein the mature transglutaminase has antimicrobial activity.

12. A pro-peptide variant of a transglutaminase pro-peptide, wherein the pro-peptide variant comprises the sequence of SEQ ID NO: 2 further comprising at least one or more amino acid modifications comprising one or more of the following:
substitution of E7 with G;
substitution of E8 with G or Y;
substitution of T9 with Q;
substitution of K10 with C, D, G, or R;
substitution of S11 with D, G, H, K, L, or Y;
substitution of Y12 with G, L, N, P, S, T, or V;
substitution of E14 with A, C, D, G, H, N, R, T, or V;
substitution of L18 with E, K, S, or W;
substitution of D21 with L or R;
substitution of D22 with G, P, Q, R, or Y;
substitution of A24 with E, H, or P;
substitution of 126 with A, E, or Y;
substitution of S38 with D, P, or Y;
deletion of Y12-P46;
deletion of L18-P46;
deletion of D22-P46; or
deletion of 126-P46.

13. The pro-peptide variant of claim 12, wherein the pro-peptide variant comprises a sequence having at least 85% amino acid sequence identity to SEQ ID NO: 2.

14. The pro-peptide variant of claim 12, wherein the pro-peptide variant comprises 5 or less amino acid substitutions with respect to SEQ ID NO: 2.

15. The pro-peptide variant of claim 12, wherein the pro-peptide variant comprises a deletion selected from:
deletion of Y12-P46;
deletion of L18-P46;
deletion of D22-P46; or
deletion of 126-P46;
wherein the pro-peptide comprises no further modifications with respect to SEQ ID NO: 2.

16. A zymogen form of a transglutaminase comprising the pro-peptide variant of claim 12.

17. The pro-peptide variant of claim 12, wherein the mature transglutaminase has antimicrobial activity.

18. A pro-peptide variant of a transglutaminase pro-peptide, wherein the pro-peptide comprises the sequence of SEQ ID NO: 2 further comprising:
(i) one or more combinations of amino acid substitutions selected from:
substitutions of D2 with S, E7 with S, and D22 with Q;
substitutions of D2 with S and E7 with S;
substitutions of D2 with I, E7 with S, and D22 with Q;
substitutions of D2 with S, E7 with S, and D21 with I;
substitutions of E7 with S and D21 with T;
substitutions of E7 with S and D21 with R;
substitutions of D2 with I, D21 with T, and D22 with Q;
substitutions of E7 with S, L18 with V, and D21 with I;
substitutions of L18 with V and D21 with T;
substitutions of E7 with S, D21 with T, and D22 with Q;
substitutions of D21 with T and D22 with Q;
substitutions of D2 with S, D21 with T, and D22 with Q;
substitutions of D2 with I, E7 with S, and D21 with I;
substitutions of D2 with S, L18 with V, and D22 with Q;
substitutions of D2 with S and D22 with Q;
substitutions of D2 with I, E2 with S, and D21 with T;
substitutions of D2 with S, E7 with S, D21 with T, and D22 with Q;
substitutions of E7 with S, L18 with V, and D21 with R;
substitutions of L18 with V, D21 with T, and D22 with Q;
substitutions of D2 with I, E7 with S, L18 with V, and D21 with R;
substitutions of E7 with S and D22 with Q; and
substitutions of D2 with I and D22 with Q; and/or
(ii) one or more amino acid substitutions comprising one or more of the following:
substitution of D2 with F;
substitution of L18 with V; or
substitution of D21 with T.

19. The pro-peptide variant of claim 18, wherein the variant comprises a sequence having at least 85% amino acid sequence identity to SEQ ID NO: 2.

20. The pro-peptide variant of claim 18, wherein the pro-peptide variant comprises 5 or less amino acid substitutions with respect to SEQ ID NO: 2.

21. A composition comprising a zymogen form of a transglutaminase and at least one pro-peptide variant selected from the pro-peptide variant according to claim 1.

22. A composition comprising a mature transglutaminase and at least one pro-peptide variant selected from the pro-peptide variant according to claim 1.

23. The composition of claim 21, wherein the transglutaminase has antimicrobial activity when in a mature form.

24. The composition of claim 22, wherein the mature transglutaminase has antimicrobial activity.

25. A composition comprising a zymogen form of a transglutaminase and at least one pro-peptide variant selected from the pro-peptide variant according to claim 7.

26. A composition comprising a mature transglutaminase and at least one pro-peptide variant selected from the pro-peptide variant according to claim 7.

27. The composition of claim 25, wherein the transglutaminase has antimicrobial activity when in a mature form.

28. The composition of claim 26, wherein the mature transglutaminase has antimicrobial activity.

29. A composition comprising a zymogen form of a transglutaminase and at least one pro-peptide variant selected from the pro-peptide variant according to claim 12.

30. A composition comprising a mature transglutaminase and at least one pro-peptide variant selected from the pro-peptide variant according to claim 12.

31. The composition of claim 29, wherein the transglutaminase has antimicrobial activity when in a mature form.

32. The composition of claim 30, wherein the mature transglutaminase has antimicrobial activity.

33. A composition comprising a zymogen form of a transglutaminase and at least one pro-peptide variant selected from the pro-peptide variant according to claim 18.

34. A composition comprising a mature transglutaminase and at least one pro-peptide variant selected from the pro-peptide variant according to claim 18.

35. The composition of claim 33, wherein the transglutaminase has antimicrobial activity when in a mature form.

36. The composition of claim 34, wherein the mature transglutaminase has antimicrobial activity.


SEQUENCE LISTING (5 sequences)

SEQ ID NO: 1 - protein, 407 aa, Streptomyces mobaraensis [sequence omitted]

SEQ ID NO: 2 - protein, 46 aa, synthetic construct
MDNGAGEETKSYAETYRLTADDVANINALNESAPAASSAGPSFRAP

SEQ ID NO: 3 - protein, 332 aa, synthetic construct
MDPDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 4 - protein, 61 aa, synthetic construct
MDNGAGEETKSYAETYRLTADDVLNINAETYRLTADDVRNINALNESAPAASSAGPSFRAP

SEQ ID NO: 5 - protein, 340 aa, synthetic construct
MDPDDRVTPPAEPLDRMPDPYRPSYGRAETVVNNYIRKWQQVYSHRDGRKSQMTEEQREWLSYGCVGVTWVNSGAYPTNRLAFASFDEDRFHNELKNGRPRSGETRAEFEGRVAKESFDEEKGFLRAREVASVMNRALENAHDESAYLDNLRRELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPMASLGAMHVYESKFRNWSEGYSDFDRGAYVITFIPKSWNTAPDKVKQGWPLEHHHHHH